(19) United States
(12) Patent Application Publication
(10) Pub. No.: US 2024/0041721 A1
(43) Pub. Date: Feb. 8, 2024

(54) METALLIC POWDER

(71) Applicant: SHISEIDO COMPANY, LTD., Tokyo (JP)

(72) Inventor: Shoji TAJIMA, Princeton Junction, NJ (US)

(73) Assignee: SHISEIDO COMPANY, LTD., Tokyo (JP)

(21) Appl. No.: 18/266,501

(22) PCT Filed: Dec. 9, 2021
(86) PCT No.: PCT/IB2021/061538

§ 371 Date: Jun. 9, 2023

Related U.S. Application Data
(60) Provisional application No. 63/127,601, filed on Dec. 18, 2020.

(51) Int. Cl. A61K 8/26; A61Q 1/12

(52) U.S. Cl. CPC A61K 8/26; A61Q 1/12; A61K 2800/436; A61K 2800/80

(57) ABSTRACT

Provided is a cosmetic composition that includes at least one micaceous pigment and at least one pearlescent pigment.

### RELATED APPLICATIONS

The present application claims priority to U.S. provisional application No. 63/127,601 filed Dec. 18, 2020 and titled “METALLIC POWDER,” which is incorporated by reference in its entirety.

### FIELD

The present disclosure relates in general to cosmetic compositions, and specifically to cosmetic compositions containing metallic powders and their methods of making and methods of use.

### SUMMARY

One embodiment is a cosmetic composition comprising at least one micaceous pigment and at least one pearlescent pigment.

### DETAILED DESCRIPTION

Unless otherwise specified “a” or “an” means one or more.

As used herein, the term “about” placed before a specific numeric value may mean±20% of the numeric value; ±18% of the numeric value, ±15% of the numeric value; ±12% of the numeric value; ±8% of the numeric value; ±5% of the numeric value; ±3% of the numeric value; ±2% of the numeric value; ±1% of the numeric value or 0.5% of the numeric value.

As used herein, the term “payoff” with respect to a cosmetic product and/or composition may refer to the product's and/or composition's color intensity/vividness and its coverage of keratinous surface(s), e.g. skin, upon applying the product and/or composition.

As used herein, the term “solid composition” may refer to a solid cosmetic composition, which may have a hardness from about 5 mm to about 1000 mm, from about 10 mm to about 800 mm, from about 15 mm to about 600 mm, from about 20 mm to about 400 mm, from about 25 mm to about 300 mm, as measured by ASTM method number 213 (Sun-Rheo Meter, Needle: 1.0 mm; weight 2 kG; hold 10.0 M; Press 20 MM/M) at ambient temperature, such as 25 degrees C.

All content information for ingredients of compositions expressed as percent (%) refers to percent (%) by mass, relative to the total mass of the composition, unless specified otherwise.

Conventional powdery compositions, such as loose powders or solidified powdery products, used in cosmetics may contain high levels of pearlescent pigments. Such conventional compositions may have a good color intensity/vividness and coverage of keratinous surfaces without a need of repeating multiple applications. However, such conventional powdery compositions, specifically when in a form of solidified compact powdery cosmetic products, may have a low stability, including an easiness to break.

Stability of solidified compact powdery compositions may be increased by adding a larger amount (usually above 10%) of conventional filler(s), such as magnesium myristate, and/or a larger amount (usually above 10%) of binder(s), such as triethyl hexanoin. However, such addition(s) may reduce shininess and color intensity of such conventional powdery compositions.

The present inventor developed a composition, which may contain a high content of pearlescent pigment(s), while having a higher stability than conventional powdery compositions with high levels of pearlescent pigment(s).

Specifically, the present inventor developed a composition, which may include at least one micaceous pigment and at least one pearlescent pigment. The composition may be in a solid form, such as a solid powder form.

The composition, such as a solid composition, e.g. a solid powdery composition, may have be stable and/or durable which may mean that the composition is being able to pass the drop test, while having a high content of spherical particles. Details of the drop test are disclosed in the Example below.

The composition, such as a solid composition, e.g. a solid powdery composition, may have a high color intensity/vividness and good coverage of keratinous surface(s), such as skin and/or lips.

The present composition, such as a solid composition, e.g. a solid powdery composition, may have a metallic finish and/or a high level of shininess.

The present composition may have a velvety feel. The present composition may provide a long lasting effect, which may mean that the composition may stay intact for a time period up to 24 hours.

The composition, such as a solid composition, e.g. a solid powdery composition, may be used in a number of color cosmetics products. In addition, applications of the present composition may include skin care products, sun care products, deodorants or hair products. In some embodiments, the composition may be a lip composition, such as a lipstick or lip balm composition. In some embodiments, the composition may be a blush composition. In some embodiments, the composition may be a foundation composition, such as a solid foundation composition. In some embodiments, the composition may be a concealer composition. In some embodiments, the composition may be an eye brow composition, such as a solid eye brow composition. In some embodiments, the composition may be an eye shadow composition. In some embodiments, the composition may be a mascara composition.

In some embodiments, the cosmetic composition may be used alone. In other words, the composition may be applied alone, without another composition, to a keratinous surface or substrate, such as skin, e.g. lips, eyelids, face, or hair, of a subject, such as a human.

In some embodiments, the composition, which may be a cosmetic composition, may be used together with another product, for example, a top coat, a primer, or a powder.

As used herein, the term “micaceous pigment” may refer to a crystal form of the pigment in a lamellar or a pallet form. Examples of micaceous pigments may include micaceous iron oxide, aluminum and graphite. Micaceous iron oxide may refer to a pigment that comprises a crystalline form of iron oxide which naturally occurs in a pallet form. In some embodiments, micaceous iron oxide may satisfy the standard of ISO 10601-2007.

In some embodiments, micaceous iron oxide may be iron oxide in a form that resembles mica, being a highly structured, layered mineral, such as crystalline Fe₂O₃. Minerals with this highly layered structure may be termed lamellar. When micaceous iron oxide are ground into smaller, finer particles, they tend to cleave along their layers, revealing flat, shiny faces that may act like tiny mirrors. These tiny mirrors may reflect UV light, protecting a material, such as a resin, in which micaceous iron oxide is incorporated from degradation and/or giving the material in which they are incorporated an attractive “sparkle”.

Micaceous pigments(s) may in a form small, thin and flaky particles. For example, micaceous pigment(s) may have an average particle thickness from about 0.05 μm to about 2 μm or from about 0.1 μm to about 1 μm or from about 0.1 μm to about 0.5 μm or any value or subrange within these ranges and an average particle size (in a plane perpendicular to the thickness) from about 3 μm to about 120 μm or from about 4 μm to about 100 μm or from about 5 μm to about 50 μm or from about 5 μm to about 30 μm or from about 10 μm to about 20 μm or any value or subrange within these ranges. In some embodiments, it may be preferred to use micaceous pigment(s) having an average particle thickness from about 0.05 μm to about 0.5 μm, such as from about 0.1 μm to about 0.3 μm, and an average particle size from about 5 μm to about 30 μm, such as from about 10 μm to about 20 μm.

The one or more micaceous pigments may provide the composition with one or more of the following properties: an increased stability, which may be determined for example, through the drop test discussed in the examples below, and/or durability, a good payoff, i.e. a color vividness/intensity which may be combined with good coverage of keratinous surface(s); a shininess and a metallic finish. In many embodiments, the one or more micaceous pigments may provide the composition with more than one, i.e. two or more, of these properties. In some embodiments, the one or more micaceous pigments may provide the composition with each of these properties. The micaceous pigments may improve stability of the composition due their physical parameters, such as an average particle thickness, an average particle size, lamellar and flat shape.

In some embodiments, a content of the one or more micaceous pigments in the composition may be from about 1.0 mass % to about 35 mass % or from about 2 mass % to about 30 mass % or from about 5 mass % to about 28 mass % or from about 10 mass % to about 25 mass % or any value or a subrange within these ranges.

In some embodiments, the one or more micaceous pigment may comprise at least one iron-containing micaceous pigment comprising iron oxide, such as an oxide of Fe^{II}, e.g. FeO or FeO₂; a mixed oxide of Fe^{II} and Fe^{III}, such as Fe₃O₄, Fe₄O₅, FesO₆, Fe₅O₇, Fe₂₅O₃₂; or an oxide of Fe^{III}, Fe₂O₃, which may be in α-Fe₂O₃ phase, β-Fe₂O₃ phase, γ-Fe₂O₃ phase, ε-Fe₂O₃ phase or a mixture of two or more of these phases. Iron oxide may constitute at least 50% or at least 60% or at least 70% or at least 80% or at least 90% of a respective iron-containing micaceous pigment.

In some embodiments, an iron-containing micaceous pigment may be such that iron oxide may be its only component. Yet in some embodiments, an iron-containing micaceous pigment may contain a non-iron oxide component in addition to iron oxide. Such non-iron oxide component may, for example, include one or more metal oxides such as alumina, titania or silica. Iron-containing micaceous pigments with alumina as a non-iron oxide component may be preferred.

In some embodiments, the at least one iron-containing micaceous pigment may comprise an iron-containing micaceous pigment comprising Fe₃O₄. In some embodiments, the at least one iron-containing micaceous pigment may comprise an iron-containing micaceous pigment comprising Fe₂O₃. Yet in some embodiments, the at least one iron-containing micaceous pigment may comprise an iron-containing micaceous pigment comprising Fe₃O₄ and an iron-containing micaceous pigment comprising Fe₂O₃, which may be in α-Fe₂O₃ phase, β-Fe₂O₃ phase, γ-Fe₂O₃ phase, α-Fe₂O₃ phase or a mixture of two or more of these phases.

Micaceous pigments, including iron-containing micaceous pigments, are disclosed, for example, in Japanese patents JP 5,400,341 and JP 5,917,293 as well as in U.S. Pat. Nos. 4,373,963 and 4,755,229, each of which is incorporated herein by reference in its entirety.

Non-limiting examples of iron-containing micaceous pigments include AM-200P and BM-200P commercially available from Titan Kogyo, Ltd. AM-200P has a lustrous, natural red color and BM-200P has a deep, silvery black color. AMP-200P includes 96% of α-Fe₂O₃ and 4% of Al₂O₃, while BM-200P includes 96% of Fe₃O₄ and 4% of Al₂O₃. Both AMP-200P and BM-200P have 10-20 μm average particle size and 0.1-0.4 μm average particle thickness.

As used herein, the term “pearlescent pigment” may refer to a pigment, which may simulate the luster of a natural pearl and optionally, provide an additional color effect, such as an angular color dependence.

In some embodiments, a content of the at least one or more pearlescent pigments in the composition may be from about 0.1 mass % to about 90 mass % or from about 10 mass % to about 90 mass % or from about 20 mass % to about 90 mass % or from about 30 mass % to about 90 mass % or from about 40 mass % to about 90 mass % or from about 50 mass % to about 90 mass % or from about 60 mass % to about 90 mass % or from about 65 mass % to about 90 mass % or from about 70 mass % to about 90 mass % or from about 75 mass % to about 90 mass % or any value or subrange within these ranges.

In some embodiments, the at least one pearlescent pigment may comprise a mica-based pearlescent pigment, which may be mica coated with one or more coating materials. Mica in the mica-based pearlescent pigment may be natural mica, synthetic mica, such as synthetic fluorophlogopite mica, or a mixture of the two. Platelets of coated mica may vary in size, shape and thickness. These characteristics together with a thickness of coating(s) may determent a color of a particular pearlescent pigment. In addition, a thickness and a coarseness of pigment particles may determine a sparkle of a particular pigment.

Non-limiting examples of coating material(s) for mica-based pearlescent pigments include metal oxides, such as titanium dioxide, iron oxides silicon dioxide, tin oxide and chromium oxide, bismuth oxychloride, ferric ferrocyanide, carmine. For example, the at least one pearlescent pigment may include one or more of the following: synthetic mica, such as synthetic fluorophlogopite mica coated with titanium oxide and iron oxide; natural mica coated with titanium oxide and silicon oxide; natural mica coated with titanium oxide and iron oxide.

In some embodiments, the at least one pearlescent pigment may comprise a non-mica-based pearlescent pigment, which may be a non-mica material coated with one or more coating materials such as those disclosed above for mica-based pearlescent pigments. Non-limiting examples of non-mica materials include calcium sodium borosilicate, alumina and silica.

In some embodiments, pearlescent pigment(s) used in the composition may have a particle size selected from about 5 μm to about 150 μm or from about 5 μm to about 125 μm or from about 10 μm to about 100 μm or from about 10 μm to about 60 μm or from about 10 μm to about 30 μm or any value or subrange within these ranges.

Pearlescent pigments are commercially available from a number of vendors, including but to limited to BASF, Sun Chemical and Merck. BASF's pearlescent pigments include but not limited to Bi-Lite® 20 pigment (natural mica coated with bismuth oxychloride), Cloisonne® Blue (natural mica coated with titanium dioxide and ferric ferrocyanide); Cloisonne® Blue Flambe (natural mica coated with iron oxides); Cloisonne® Cerise Flambe (natural mica coated with iron oxides); Cloisonne® Copper (natural mica coated with iron oxides and titanium dioxide); Cloisonne® Gold (natural mica coated with iron oxides and titanium dioxide); Cloisonne® Golden Bronze (natural mica coated with iron oxides and titanium dioxide); Cloisonne® Green (natural mica coated with iron oxides and chromium oxide); Cloisonne® Nu-Antique Blue (natural mica coated with titanium dioxide, iron oxides and ferric ferrocyanide); Cloisonne® Nu-Antique Bronze (natural mica coated with iron oxides and titanium dioxide); Cloisonne® Nu-Antique Copper (natural mica coated with iron oxides and titanium dioxide); Cloisonne® Nu-Antique Gold (natural mica coated with iron oxides and titanium dioxide); Cloisonne® Nu-Antique Bronze (natural mica coated with titanium dioxide, iron oxides and chromium oxide); Cloisonne® Nu-Antique Rouge Flambe (natural mica coated with iron oxides and titanium dioxide); Cloisonne® Orange (natural mica coated with titanium dioxide and iron oxides); Cloisonne® Regal Gold (natural mica coated with titanium dioxide and iron oxides); Cloisonne® Rouge Flambe (natural mica coated with iron oxides and titanium dioxide); Cloisonne® Satin Bronze (natural mica coated with iron oxides); Cloisonne® Super Blue (natural mica coated with titanium dioxide and ferric ferrocyanide); Cloisonne® Super Bronze (natural mica coated with iron oxides); Cloisonne® Super Gold (natural mica coated with titanium dioxide and iron oxides); Cloisonne® Super Green (natural mica coated with titanium dioxide, iron oxides and ferric ferrocyanide); Cloisonne® Super Red (natural mica coated with titanium dioxide and carmine); Flamenco® Blue (natural mica coated with titanium dioxide); Flamenco® Gold (natural mica coated with titanium dioxide); Flamenco® Green (natural mica coated with titanium dioxide); Flamenco® Orange (natural mica coated with titanium dioxide); Flamenco® Pearl (natural mica coated with titanium dioxide); Flamenco® Satin Pearl (natural mica coated with titanium dioxide); Flamenco® Satina (natural mica coated with titanium dioxide); Flamenco® Summit Magenta (natural mica coated with titanium dioxide); Reflecks™ Gilded Gold (calcium sodium borosilicate coated with titanium dioxide and iron oxides), Timica® Nu-Antique bronze (natural mica coated with iron oxides and titanium dioxide).

Sun Chemical's pearlescent pigments include but are not limited to SunSHINE® Crystal Golden (INCI name: Synthetic Fluorphlogopite (and) Titanium Dioxide (and) Iron Oxide); C91-4236 INTENZA® FLAMINGO (Synthetic Fluorphlogopite & Ci 77891 & Ci 73360); C91-4221 INTENZA® ORANGE ZEST(Synthetic Fluorphlogopite & Ci 77891 & Ci 15850); INTENZA® MANGO TANGO C91-1221(Mica & Ci 77891 & Ci 15850). Merck's pearlescent pigments include but not limited to those sold under tradenames Ronastar® and Timiron®. Timiron® pearlescent pigments include but not limited to Timiron® Super Color pearlescent pigments, Timiron® Splendid Color pearlescent pigments and Timiron® SynBeam Colors, all of which have an average particle size between about 10 microns and about 60 microns. Timiron® Super Color pearlescent pigments include natural mica coated with titanium oxide pearlescent pigments, such as Timiron® Super Green, Timiron® Super Blue, and Timiron® Super Red, and natural mica coated with titanium oxide and tin oxide pearlescent pigments, such as Timiron® Super Copper and Timiron® Super Violet. Timiron® Splendid Color pearlescent pigments are natural mica coated with titanium oxide and silicon oxide pearlescent pigments, such as Timiron® Splendid Gold, Timiron® Splendid Red, Timiron® Splendid Blue, Timiron® Splendid Green, Timiron® Splendid Copper, Timiron® Splendid Violet. Timiron® SynBeam Color pearlescent pigments are synthetic mica (synthetic Fluorphlogopite) coated with titanium oxide and tin oxide, such as Timiron® SynBeam Red, Timiron® SynBeam Blue and Timiron® SynBeam Violet.

In some embodiments, a ratio between a mass content of the at least one micaceous pigment and a mass content of the least one pearlescent pigment in the composition may be from about 1:2 to about 1:30 or from about 1:2 to about 1:20 to about 1:2 to about 1:10 or from about 1:2.5 to about 1:6 or from about 1:3 to about 1:5 or any value or subrange within these ranges.

In some embodiments, the composition, such as a solid composition, e.g. a solid powder composition, may contain at least one filler, which is not a micaceous material, a micaceous pigment or a pearlescent pigment. In some embodiments, a content of such at least one filler may be from about 0.1 mass % to about 80 mass %. Yet in some embodiments, a content of the at least one filler in the present composition may be lower compared to a filler content in conventional powder compositions used for cosmetics. For example, in some embodiments, a content of the at least one filler in the present composition may be from about 0.1 mass % to about 6 mass % or from about 0.5 mass % to about 5 mass % or from about 1 mass % to about 4 mass % or any values or subrange within these ranges.

The filler(s) may be of mineral or organic nature. In some embodiments, the filler(s) may provide softness, a matt effect and/or uniformity. The filler(s) may have a different variety of shapes and structure; they may be of lamellar, globular, regular, irregular form, fibers or in any intermediate form. The filler(s) may be surface uncoated or coated. Non-limiting examples of coatings for the filler(s) may include a butter, such as shea butter, cocoa butter, cocoa seed butter, mango seed butter. In some embodiments, the fillers may be selected from but not limited to mica, which may be natural or synthetic, uncoated or coated, for example, with a butter, such as shea butter, cocoa butter, cocoa seed butter, mango seed butter; starches; chlorphenesin powder kaolin, bentone, silica, nylon, such as nylon-12, and zinc myristate. In some embodiments, examples of filler(s) may include but are not limited to mica, which may or may not be coated, zinc myristate, silica, nylon-12, fluorphlogopite and chlorphenesin.

In some embodiments, the composition, such as a solid composition, e.g. a solid powdery composition, may contain at least one binder, which is not a micaceous material, a micaceous pigment or a pearlescent pigment. A content of such at least one binder in the present composition may be lower compared to a binder content in conventional powder compositions used for cosmetics. In some embodiments, a content of the at least one binder in the present composition may be from about 0.1 mass % to about 25 mass % or from about 1 mass % to about 20 mass % or from about 2 mass % to about 15 mass % or from about 3 mass to about 13 mass % or any values or subrange within these ranges.

Non-limiting examples of binder(s) caprylic/capric triglyceride, polyglyceric-3 diisostearate, sorbitan sesquilisostearte, dimethicone, phenyltrimethicone, shea butter, jojoba esters, octyldodecanol.

In some embodiments, the composition may include only (a) at least micaceous pigment; (b) at least one pearlescent pigment; (c) at least one filler (optional) and (d) at least one binder (optional). In other words, the composition may include no other ingredients that (a), (b), (c) and (d). Yet in some other embodiments, the composition may include one or more ingredients other than ingredients (a), (b), (c) and (d), such as actives, vitamins, extracts.

In some embodiments, the composition may be anhydrous and contain less than about 1% or less than about 0.5% or less than about 0.3% or less than about 0.1% of water or no water at all.

In some embodiments, a total content of ingredient(s) (such as filler(s) and/or binder(s)), of the composition, which are not a micaceous pigment or a pearlescent pigment, is no more than about 20 mass % or no more than about 18 mass % or no more than mass 16% or no more than 14 mass % or no more than 12 mass % or no more than 10 mass % or no more than 8 mass % or any value or subrange within these ranges.

In some embodiments, the present composition, such as a solid composition, e.g. a solid powdery composition, may be talc free, i.e. it may contain no talc at all.

In some embodiments, the present composition, such as a solid composition, e.g. a solid powdery composition, may be free of microsized plastic beads, i.e. it may contain no plastic beads having a size from 0.5 microns to 100 microns or from 1 micron to 100 microns at all.

Embodiments described herein are further illustrated by, though in no way limited to, the following working examples.

### Example

### Production Method of Solid Powder Cosmetic

Powder components and oil components, which have been melted by heating as necessary, described in the formulations of the table below were mixed with a Henschel mixer to obtain a uniform mixture. To the mixture, iso-paraffin was added, and a slurry was obtained by mixing with a dispersion mixer. The slurry was filled into an inner plate, and the solvent was removed by suction, and then drying was carried out to obtain a solid powder cosmetic.

### Evaluation of Solid Powder Cosmetic

### (a) Drop Test (Impact Resistance)

A solid powdery composition was placed in a cosmetic container, such as a compact, and the number of times until breakage was examined by dropping it on a metal plate, from the height of 30 cm, in a state that the surface of the composition is facing downward. The number of test samples were (N)=3 for the same batch. Compositions were evaluated to have satisfactory impact resistance if the average drop number was at least 5, and to have excellent impact resistance if the average drop number was 6 or higher.

### (b) Evaluation

Ten expert panelists of cosmetics were asked to apply a solid powdery composition on the skin and to evaluate, in 5-scale rating (between very poor usability: 0 and very good usability-payoff: 5), for “(on the skin) shininess and 3-scale rating for payoff. The average evaluation value was calculated, and the judgement was made as described below and shown with symbols in the Tables below.

TABLE 1

| Function | Trade name | INCI Name | Exp. 01 | Exp. 02 |
|---|---|---|---|---|
| Filler | BUTTERPOWDER MCB91-Q | MICA & THEOBROMA CACAO (COCOA) SEED | 1.70 | 1.70 |
| | | BUTTER TOCOPHERYL ACETATE & QUERCETIN | | |
| | ZINC MYRISTATE | ZINC MYRISTATE | 1.08 | 1.08 |
| | (POWDER BASE M) | | | |
| | ELESTAB ® CPN | CHLORPHENESIN | 0.20 | 0.20 |
| | AM-200P | IRON OXIDES & ALUMINA | 20.00 | |
| Pearl | SUNSHINE CRYSTAL | SYNTHETIC FLUORPHLOGOPITE & TITANIUM | & 35.00 | 35.00 |
| Pigments | GLDN C843512 | DIOXIDE IRON OXIDES | | |
| | TIMIRON ® SPLENDID GOLD | TITANIUM DIOXIDE & MICA & SILICA | 30.00 | 30.00 |
| | CLOISONNE ROUGE FLAMBE | MICA & IRON OXIDES & TITANIUM DIOXIDE | | 20.00 |
| Binder | MYRITOL 318 | CAPRYLIC/CAPRIC TRIGLYCERIDE | 6.00 | 6.00 |
| | PLUROL DIISOSTEARIQUE CG | POLYGLYCERYL-3 DIISOSTEARATE | 5.00 | 5.00 |
| | #5793 | | | |
| | ESTEMOL 182V | SORBITAN SESQUIISOSTEARATE | 1.00 | 1.00 |
| | VITAMIN E-ACETATE CARE 30499500 | TOCOPHERYL ACETATE | 0.02 | 0.02 |
| | | | 100.00 | 100.00 |
| | | Shininess | +++++ | +++ |
| | | Pay off | +++ | +++ |
| | | Drop test(N = 3) | 6.3 | 4.3 |

Shinniness
+++++ Excellent
++++ very good
+++ good
++ poor
+ very poor
Pay off
+++ Very good
++ good
+ poor

The data in Table 1 shows that Removal of a micaceous pigment, AM-200P, and replacement with another pearlescent pigment reduces stability of a pressed powder and also reduces shinny finish. The data in Table 1 indicates that adding a micaceous pigment, such as AM-200P, increases stability of compositions.

TABLE 2

| Function | Trade Name | INCI Name | Exp. 01 | Exp. 03 | Exp. 04 | Exp. 05 | Exp. 06 |
|---|---|---|---|---|---|---|---|
| Filler | BUTTERPOWDER | MICA & THEOBROMA CACAO | 1.70 | 1.70 | 1.70 | 1.70 | 1.70 |
| | MCB91-Q | (COCOA) SEED BUTTER & | | | | | |
| | | TOCOPHERYL ACETATE & | | | | | |
| | | QUERCETIN | | | | | |
| | ZINC MYRISTATE | ZINC MYRISTATE | 1.08 | 1.08 | 1.08 | 1.08 | 1.08 |
| | (POWDER BASE M) | | | | | | |
| | ELESTAB ® CPN | CHLORPHENESIN | 0.20 | 0.20 | 0.20 | 0.20 | 0.20 |
| | AM-200P | IRON OXIDES & ALUMINA | 20.00 | | | | |
| | MICA PDM-9L-20 | SYNTHETIC | | 20.00 | | | |
| | | FLUORPHLOGOPITE | | | | | |
| | MAGNESIUM MYRISTATE | MAGNESIUM MYRISTATE | | | 20.00 | | |
| Pearl | SUNSHINE CRYSTAL | SYNTHETIC | 35.00 | 35.00 | 35.00 | 35.00 | 45.00 |
| Pigments | GLDN C843512 | FLUORPHLOGOPITE & | | | | | |
| | | TITANIUM DIOXIDE & | | | | | |
| | | IRON OXIDES | | | | | |
| | TIMIRON ® SPLENDID | TITANIUM DIOXIDE & | 30.00 | 30.00 | 30.00 | 30.00 | 40.00 |
| | GOLD | MICA & SILICA | | | | | |
| | CLOISONNE ROUGE | MICA & IRON OXIDES & | | | | | |
| | FLAMBE | TITANIUM DIOXIDE | | | | | |
| | Cloisonne ® Satin Rouge | MICA & IRON OXIDES | | | | 20.00 | |
| | 450M | | | | | | |
| Binder | MYRITOL 318 | CAPRYLIC/CAPRIC | 6.00 | 6.00 | 6.00 | 6.00 | 6.00 |
| | | TRIGLYCERIDE | | | | | |
| | PLUROL | POLYGLYCERYL-3 | 5.00 | 5.00 | 5.00 | 5.00 | 5.00 |
| | DIISOSTEARIQUE CG | DIISOSTEARATE | | | | | |
| | #5793 | | | | | | |
| | ESTEMOL 182V | SORBITAN | 1.00 | 1.00 | 1.00 | 1.00 | 1.00 |
| | | SESQUIISOSTEARATE | | | | | |
| | VITAMIN E-ACETATE | TOCOPHERYL ACETATE | 0.02 | 0.02 | 0.02 | 0.02 | 0.02 |
| | CARE 30499500 | | | | | | |
| | | | 100.00 | 100.00 | 100.00 | 100.00 | 100.00 |
| | | Shininess | +++++ | ++ | + | ++ | +++ |
| | | Pay off | +++ | +++ | + | ++ | +++ |
| | | Drop test(N = 3) | 6.3 | 4.7 | >10 | 5.3 | 3.7 |

Table 2 demonstrates that compositions using conventional fillers, such as magnesium myristate and mica, instead of micaceous pigment(s), such as AM-200P, may not perform well enough to achieve both shinny finish and good stability. The data in Table 2 also demonstrates that compositions without micaceous pigment(s), such as AM-200P, do not possess the same combination of excellent shininess, very good payoff, excellent stability (through the drop test) as the compositions containing micaceous pigment(s), such as AM-200P.

TABLE 3

| | | Particle range | Average Particle | Thickness | |
|---|---|---|---|---|---|
| | | (μm) | (μm) | (μm) | Description |
| AM-200P | IRON OXIDES & ALUMINA | 10~20 | — | 0.1-0.3 | Micaceous pigment |
| CLOISONNE ROUGE | MICA & IRON OXIDES & | 8~39 * | — | N/D | Conventional pearl pigment |
| FLAMBE | TITANIUM DIOXIDE | | | | |
| MICA PDM-9L-20 | SYNTHETIC | N/D | 19.8 | 0.2-0.3 | Conventional filler |
| | FLUORPHLOGOPITE | | | | |
| CLOISONNE SATIN | MICA & IRON OXIDES | 4~18 * | — | N/D | Conventional small particle |
| ROUGE 450M | | | | | sized pearl pigment |
| MAGNESIUM | MAGNESIUM MYRISTATE | | | | Conventional filler |
| MYRISTATE | | | | | |

TABLE 4

| | Raw Material | INCI Name | Exp. 01 | Exp. 07 | Exp. 08 | Exp. 02 |
|---|---|---|---|---|---|---|
| Filler | BUTTERPOWDER | MICA & THEOBROMA CACAO | 1.70 | 1.70 | 1.70 | 1.70 |
| | MCB91-Q | (COCOA) SEED BUTTER & | | | | |
| | | TOCOPHERYL ACETATE & | | | | |
| | | QUERCETIN | | | | |
| | ZINC MYRISTATE | ZINC MYRISTATE | 1.08 | 1.08 | 1.08 | 1.08 |
| | (POWDER BASE M) | | | | | |
| | ELESTAB ® CPN | CHLORPHENESIN | 0.20 | 0.20 | 0.20 | 0.20 |
| | AM-200P | IRON OXIDES & ALUMINA | 20.00 | 10.00 | 5.00 | |
| Pearl | SUNSHINE CRYSTAL | SYNTHETIC FLUORPHLOGOPITE & | 35.00 | 35.00 | 35.00 | 35.00 |
| Pigments | GLDN C843512 | TITANIUM DIOXIDE & | | | | |
| | | IRON OXIDES | | | | |
| | TIMIRON ® SPLENDID | TITANIUM DIOXIDE & MICA & | 30.00 | 30.00 | 30.00 | 30.00 |
| | GOLD | SILICA | | | | |
| | CLOISONNE ROUGE | MICA & IRON OXIDES & TITANIUM | | 10.00 | 15.00 | 20.00 |
| | FLAMBE | DIOXIDE | | | | |
| Binder | MYRITOL 318 | CAPRYLIC/CAPRIC TRIGLYCERIDE | 6.00 | 6.00 | 6.00 | 6.00 |
| | PLUROL | POLYGLYCERYL-3 | 5.00 | 5.00 | 5.00 | 5.00 |
| | DIISOSTEARIQUE CG | DIISOSTEARATE | | | | |
| | #5793 | | | | | |
| | ESTEMOL 182V | SORBITAN SESQUIISOSTEARATE | 1.00 | 1.00 | 1.00 | 1.00 |
| | VITAMIN E-ACETATE | TOCOPHERYL ACETATE | 0.02 | 0.02 | 0.02 | 0.02 |
| | CARE 30499500 | | | | | |
| | | | 100.00 | 100.00 | 100.00 | 100.00 |
| | | Shininess | +++++ | ++++ | ++++ | +++ |
| | | Pay off | +++ | +++ | +++ | +++ |
| | | Drop test(N = 3) | 6.3 | 5.3 | 5 | 4.3 |

Table 4 provides evidence that adding a micaceous pigment, such AM-200P, improves stability and that at least 5% of a micaceous pigment, such as AM-200P, may improve both stability and shiny appearance.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A cosmetic composition comprising at least one micaceous pigment and at least one pearlescent pigment.

2. The cosmetic composition of claim 1, wherein a content of the at least one micaceous pigment is from about 1.0 mass % to about 35 mass %.

3. The cosmetic composition of claim 1, wherein a content of the at least one pearlescent pigment is from about 0.1 mass % to about 90 mass %.

4. The cosmetic composition of claim 3, wherein the content of the at least one pearlescent pigment is at least 70 mass %.

5. The cosmetic composition of claim 1, wherein the least one micaceous pigment comprises at least one iron-containing micaceous pigment comprising iron oxide.

6. The cosmetic composition of claim 5, wherein the iron oxide is Fe₂O₃.

7. The cosmetic composition of claim 5, wherein the iron oxide is Fe₃O₄.

8. The cosmetic composition of claim 5, wherein the at least one iron-containing micaceous pigment further comprises alumina.

9. The cosmetic composition of claim 1, wherein the at least one pearlescent pigment comprises at least mica-based pigment comprising mica coated with one or more metal oxides.

10. The cosmetic composition of claim 9, wherein the mica is natural mica.

11. The cosmetic composition of claim 9, wherein the mica is synthetic mica.

12. The cosmetic composition of claim 9, wherein the one or more metal oxides are selected from titanium oxide, iron oxide and silicon oxide.

13. The cosmetic composition of claim 9, wherein the at least mica-based pigment comprises synthetic mica coated with titanium oxide and iron oxide.

14. The cosmetic composition of claim 9, wherein the at least mica-based pigment comprises natural mica coated with titanium oxide and silicon oxide.

15. The cosmetic composition of claim 9, wherein the at least mica-based pigment comprises natural mica coated with titanium oxide and iron oxide.

16. The cosmetic composition of claim 1, which is a stable composition.

17. The cosmetic composition of claim 1 further comprising at least one filler.

18. The cosmetic composition of claim 17, wherein a content of the at least one filler in the composition is from about 0.1 mass % to about 80 mass %.

19. The cosmetic composition of claim 1 further comprising at least one binder.

20. The cosmetic composition of claim 19, wherein a content of the at least one binder in the composition is from about 0.1 mass % to about 25 mass %.

21. A cosmetic method comprising applying the cosmetic composition of claim 1 to a keratinous surface.
